(19) United States
(12) Patent Application Publication
(10) Pub. No.: US 2010/0268016 A1
(43) Pub. Date: Oct. 21, 2010

(54) Magnetic Orthopedic Device

(76) Inventor: Joyce Walker, Bay City, MI (US)

Correspondence Address: JAY M. SCHLOFF, 6960 Orchard Lake Road, Suite 315, West Bloomfield, MI, 48322, US

(21) Appl. No.: 12/762,174

(22) Filed: Apr. 16, 2010

Related U.S. Application Data
(60) Provisional application No. 61/169,997, filed on Apr. 16, 2009.

(51) Int. Cl. A61N 2/00

(52) U.S. Cl. 600/15

(57) ABSTRACT

A magnetic orthopedic device comprises for covering a body region in which a person is experiencing pain. In an embodiment, the device comprises an elongate band of fabric with complimentary fastener elements disposed at opposite ends of thereof. The band may secure the device in the proximity of or around a body part or region, such as an elbow, torso, or lower back. The device further comprises at least one removably attachable section of material, which section includes a plurality of magnets disposed thereon (and preferably, hematite magnets.) The attachable section of material may be disposed at various points along the elongate band while the band is attached to a user's body, which allows a user to selectively position the magnets against a particular area of the body that is experiencing pain.

### CROSS-REFERENCE TO RELATED APPLICATIONS

The present disclosure claims priority under 35 United States Code, Section 119 on the U.S. Provisional Patent Application No. 61/169,997 filed on Apr. 16, 2009, the disclosure of which is incorporated by reference.

### FIELD OF THE DISCLOSURE

The present disclosure relates to devices for treating bodily pain, and more particularly, to a device that treats bodily pain through magnetism and a device that is adjustable such that it can fit around a variety of body parts and can be variably placed on particular body areas or sections that are experiencing pain.

### BACKGROUND

As people engage in physical activity, their bodies may be subjected to physical impact that may damage muscles, joints, and the overall skeletal system. As people age, their bodies may deteriorate gradually such that they are not able to sustain the same level of physical activity and stress that they once were. In either case, the bodies may incur pain as a result of such physical impact, activity, and stress, and a person will want to treat the pain to lessen its negative effect on the body and the person's overall disposition.

Existing treatments include pharmaceutical regimens, physical therapy (including chiropractic treatment), and in some cases, surgery. Each of these options is costly, time-consuming, and physically intrusive, and as such, not necessarily desirable for the person suffering from pain.

Accordingly, what is needed is a low-cost, non-intrusive, and quick, yet still effective system and method of treatment of pain to one's body. There also exists a need for a system and method of pain treatment that is easy for the user to administer to him- or herself.

Therefore, it is an object of the present disclosure to obviate above and other disadvantages from existing art and to provide for a magnetic orthopedic device that overcomes all of the disadvantages of the prior art, while still maintaining the advantages of the prior art.

It is also an object of the present disclosure to provide for a system and method of relieving and treating joint and other orthopedic maladies of a person, and of increasing a person's energy level.

### SUMMARY OF THE DISCLOSURE

The present disclosure provides for a magnetic orthopedic device for covering a body region in which a person is experiencing pain. In an embodiment, the magnetic orthopedic device comprises an elongate band of fabric. Complimentary fastener elements disposed at opposite ends of the band may secure the device in the proximity of or around a body part or region, such as an elbow, torso, or lower back, for example. The fastener elements may comprise a hook and loop arrangement, a Velcro® arrangement, a snap-fit arrangement, a clip arrangement, and the like. The band may further include a belt-loop on one of its elongate ends for receiving the other elongate end of the band. In an embodiment, the width of the band may taper at each elongate end thereof to provide ease and comfort when the wearer has secured the device around a body part or region.

The device further comprises at least one removably attachable section of material, which section includes a plurality of magnets disposed thereon (and which section will hereinafter be referred to as “magnet section.”). The at least one magnet section may comprise a substantially quadrilateral area of fabric that extends a portion of the length of the band of fabric. The magnet section may further be configured in its width dimension such that the width dimension is no greater than the width dimension of the band of fabric. The magnets may be hematite magnets.

### BRIEF DESCRIPTION OF THE DRAWINGS

The advantages and features of the present disclosure will become better understood with reference to the following detailed description and claims taken in conjunction with the accompanying drawings, wherein like elements are identified with like symbols, and in which:

FIG. 1 illustrates a perspective view of a magnetic orthopedic device, in accordance with one embodiment of the present disclosure; and

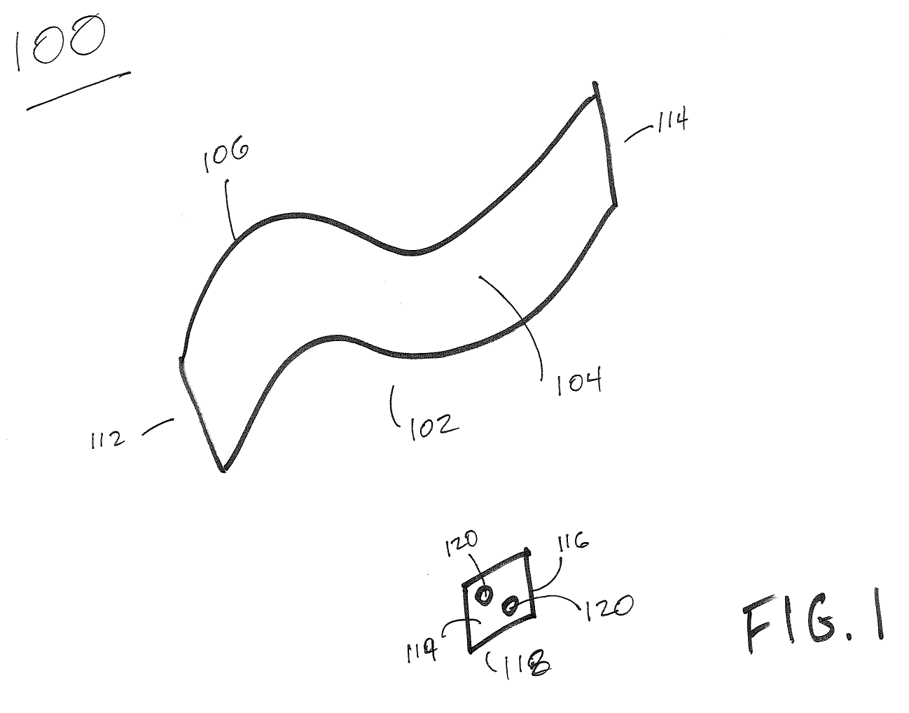

FIG. 2 illustrates a perspective view of a magnetic orthopedic device with a magnet section thereof attached to an elongate band thereof, in accordance with one embodiment of the present disclosure;

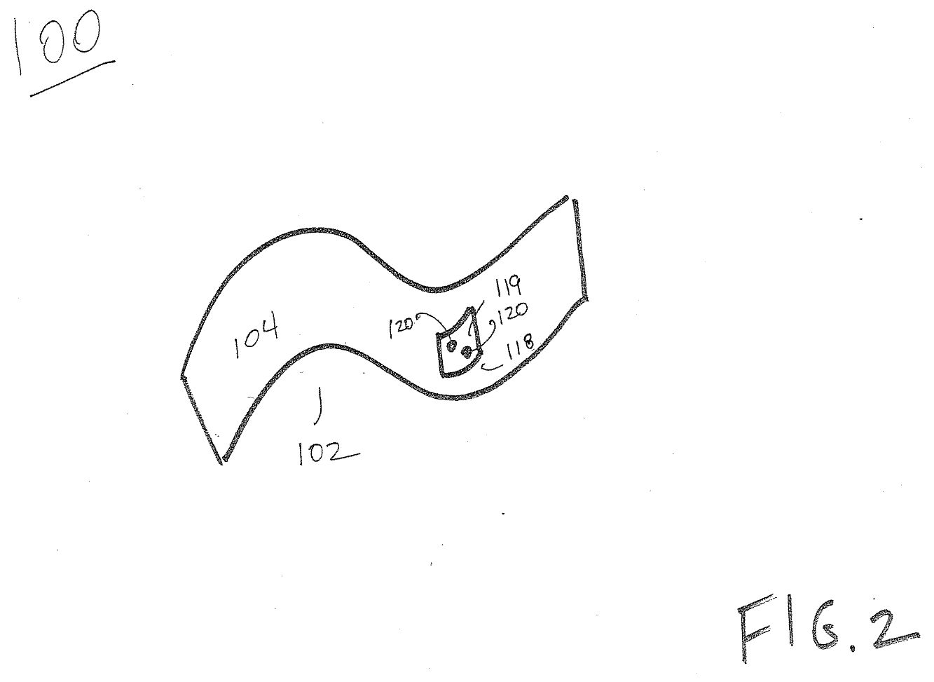

### Like reference numerals refer to like parts throughout the description of several views of the drawings.

### DETAILED DESCRIPTION OF THE DISCLOSURE

The best mode for carrying out the disclosure is presented in terms of its preferred embodiment. The preferred embodiments described here in detail for illustrative purposes are subject to many variations. It is understood that various omissions, substitutions of equivalents are contemplated as circumstances may suggest or render expedient, but is intended to cover the application or implementation without departing from the spirit or scope of the claims of the present disclosure. As used herein, the terms “a” and “an” herein do not denote a limitation of quantity, but rather denote the presence of at least one of the referenced item.

The present disclosure provides for a magnetic orthopedic device 100 for covering a body region in which a person is experiencing pain. In an embodiment, the magnetic orthopedic device 100 comprises an elongate band of fabric material 102 with complimentary fastener elements disposed at a first end 112 and second end 114 thereof. The fastener elements may secure the device in the proximity of or around a body part or region, such as an elbow, torso, or lower back, for example. The fastener elements may comprise a hook and loop arrangement, a Velcro® arrangement, a snap-fit arrangement, a clip arrangement, and the like. The band may further include a belt-loop on one of the first and second end thereof ends for receiving the other end of the band. In an embodiment, the width of the band may taper at each first and second end thereof to provide ease and comfort when the wearer has secured the device around a body part or region.

The device further comprises at least one removably attachable section of material 118, which section includes a plurality of magnets 120 disposed thereon (and which section will hereinafter be referred to as “magnet section.”) The magnet section 118 comprises a first side 116 and a second side 119.

The band of fabric 102 further comprises at first side 106 and a second side 104. The first side 106 may be distal to a wearer's body when the length of fabric is secured on the wearer's body. The second side 104 may be proximate to the wearer's body when the band of fabric is secured on the wearer's body. The band of fabric may be comprised of a stretchable, resilient, and washable material such as foam rubber, neoprene, Lycra®, nylon, and the like. At least one of the first 106 and second 104 sides of the elongate band 102 may be adorned with designs that suit a wearer's preference, such as pictures of animals, vehicles, landscapes, and the like. It will be understood that the elongate band 102 may be produced in solid colors or in a variety of stylized designs, as well.

A complimentary fastener arrangement may be disposed along at least a portion of the second side 104 of the band of fabric 102. In an embodiment, the fastener arrangement may be a hook and loop arrangement or a Velcro® material. This material may be disposed on the second side 104 of the length of fabric by way of stitching, heat-sealing, and the like. The fastener arrangement on the second side 104 of the band of fabric 102 will be capable of receiving at least one magnet section. In an embodiment, a wearer may attach a plurality of magnet sections 118 to the second side of the band of fabric, at locations along said second side 104 of the band 102 that may, when the device 100 is secured around the wearer's body, abut the body areas that the wearer wishes to subject to the magnets and the therapeutic benefits thereof.

In an embodiment, the at least one magnet section 118 comprises a substantially quadrilateral area of fabric that is extends approximately one-tenth of the length of the band of fabric. The magnet section may further be configured in its width dimension such that the width dimension is no greater than the width dimension of the band of fabric.

In an embodiment, the at least one magnet section 118 comprises a first side 116 with a fastener arrangement that is complimentary with the fastener arrangement of the second side 104 of the elongate band of fabric 102, and a second side 119 on which a plurality of magnets 120 is disposed. The magnets 120 may be disposed on and secured to the second side 119 of the magnet section 118 by way of an adhesive such as glue or epoxy, or may be sewn into or snapped on the second side 119. The magnets 120 may be disposed in any fashion or arrangement, such as, but not limited to, a linear or circular array. In an embodiment, the magnets 120 may be hematite magnets.

In use, a wearer may attach the at least one magnet section 118 to the second side 104 of the elongate band 102 and thereafter secure the elongate band 102 to his or her body. The size of the at least one magnet section 118 allows a wearer to attach a magnet section 118 at a variety of locations along the length of the band of fabric 102. Accordingly, a wearer may attach a magnet section 118 at a point along the length of the band of fabric 102 such that the magnet section 118 may be adjacent to a body part that is causing pain to the user. Furthermore, the size of the magnet section 118 allows a wearer to attach a plurality of magnet sections 118 along the length of the band of fabric 102, such that a wearer may have the plurality of magnet sections 118 adjacent to more than one body part while wearing the device 100. Once a wearer has attached at least one magnet section 118 to the second side 104 of the elongate band 102, the wearer may thereafter secure the elongate band 102 on an area of his or her body.

The magnetic orthopedic device 100 disclosed herein allows the wearer to adjustably position and dispose an array of magnets 120 against a body area or areas that is or are experiencing pain. The positioning of the magnets 120 against or adjacent to such body area or areas facilitates relief of such pain by way of the magnets' 120 capability of relieving pain. The magnets 120 may also encourage increased blood circulation and oxygen intake in a wearer's body, reduce inflammation in a wearer's body, and may decrease healing and recovery times of a wearer's body's injured or fatigued body parts.

The present disclosure offers advantages over other devices, in that the present disclosure facilitates pain relief without the ingestion of pharmaceutical compounds, harshness or cost of physical therapy, or the cost and intrusion of surgery. The disclosure further allows a wearer to precisely position the magnet sections 118 to maximize the relief and benefits provided by the device 100. The device 100 is also lightweight, customizable in size and positioning on a body part or area, and is washable for hygienic and aesthetic purposes. The size and lightweight qualities of the device 100 permit a wearer to employ the device comfortably for extended periods to increase relief.

It will be obvious to one skilled in the art that the magnetic orthopedic device 100 may be of various sizes to accommodate the varying sizes of particular wearers' body parts, extremities and the like.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present disclosure

What is claimed is:

1. A magnetic orthopedic device, the device comprising:
an elongate band of fabric material, said elongate band having a first end and a second end, said elongate band having complementary fastener elements at said first end and second end thereof, and said elongate band further having a first side and a second side,
wherein the second side comprises a fastener arrangement disposed on at least a portion of the second side,
at least one section of material with a first side and a second side,
wherein the first side of the at least one section of material comprises a fastener arrangement disposed on at least a portion of the said first side, which fastener arrangement of said first side is complementary to the fastener arrangement disposed on said second side of the elongate band, such that the said at least one section of material is removably attachable to said elongate band, and
wherein said second side of the at least one said section of material comprises a plurality of magnets disposed thereon.

2. The magnetic orthopedic device of claim 1, wherein the magnets comprise hematite magnets.

3. The magnetic orthopedic device of claim 1, wherein the complementary fastener elements for the first end and second end of the elongate band comprise one of a hook and loop arrangement, a Velcro® arrangement, a snap-fit arrangement, a clip arrangement.

4. The magnetic orthopedic device of claim 1, wherein the length dimension of the at least one section of material is approximately one-tenth of the length dimension of the elongate band.

5. The magnetic orthopedic device of claim 1, wherein the complementary fastener arrangements of the at least one section of material and the second side of the elongate band comprises a hook and loop arrangement.

6. The magnetic orthopedic device of claim 1, wherein the elongate band further comprises a belt loop disposed around the band for receiving an end of the band therethrough.
